(19) United States
(12) Patent Application Publication
(10) Pub. No.: US 2014/0214123 A1
(43) Pub. Date: Jul. 31, 2014

(54) Bilateral Communication in a Two-Channel System

(71) Applicants: Jan Janssen, New South Wales (AU); Erika Van Baelen, Heverlee (BE); Koen Van den Heuvel, HOVE (BE); Werner Meskens, Opwijk (BE)

(72) Inventors: Jan Janssen, New South Wales (AU); Erika Van Baelen, Heverlee (BE); Koen Van den Heuvel, HOVE (BE); Werner Meskens, Opwijk (BE)

(21) Appl. No.: 13/751,768

(22) Filed: Jan. 28, 2013

(51) Int. Cl. A61N 1/36

(52) U.S. Cl. CPC A61N 1/36032
USPC 607/57

(57) ABSTRACT

A hearing prosthesis system has a hearing prosthesis device for each ear. In the case of a cochlear implant, the prosthesis may have both an external portion and an internal portion. The external portion provides a wireless communication to the internal portion. Additionally, each prosthesis is configured to communicate with the other prosthesis. Thus, a second wireless signal provides a bilateral communication link between the two prostheses. Advantageously, in order to mitigate potential wireless interference due to each prosthesis communicating with both a corresponding internal component and the other external component, the disclosed systems and methods operate in a specific communication mode. Under this mode, for each time period, one external component is in a transmit mode, while the other external component is in a receive mode. As the time period advances, the external components simultaneously switch modes.

### BACKGROUND

Various types of hearing prostheses provide people having different types of hearing loss with the ability to perceive sound. Hearing loss may be conductive, sensorineural, or some combination of both conductive and sensorineural hearing loss. Conductive hearing loss typically results from a dysfunction in any of the mechanisms that ordinarily conduct sound waves through the outer ear, the eardrum, or the bones of the middle ear. Sensorineural hearing loss typically results from a dysfunction in the inner ear, including the cochlea, where sound vibrations are converted into neural signals, or any other part of the ear, auditory nerve, or brain that process the neural signals.

People with some forms of conductive hearing loss may benefit from hearing prostheses, such as acoustic hearing aids or vibration-based hearing aids. An acoustic hearing aid typically includes a small microphone to detect sound, an amplifier to amplify certain portions of the detected sound, and a small speaker to transmit the amplified sound into the person's ear. Vibration-based hearing aids typically include a small microphone to detect sound, and a vibration mechanism to apply vibrations corresponding to the detected sound to a person's bone, thereby causing vibrations in the person's inner ear, thus bypassing the person's auditory canal and middle ear. Vibration-based hearing aids include bone anchored hearing aids, direct acoustic cochlear stimulation devices, or other vibration-based devices.

A bone anchored hearing aid typically utilizes a surgically-implanted mechanism to transmit sound via direct vibrations of the skull. Similarly, a direct acoustic cochlear stimulation device typically utilizes a surgically-implanted mechanism to transmit sound via vibrations corresponding to sound waves to generate fluid motion in a person's inner ear. Other non-surgical vibration-based hearing aids use similar vibration mechanisms to transmit sound via direct vibration of teeth or other cranial or facial bones.

Persons with certain forms of sensorineural hearing loss may benefit from prostheses, such as cochlear implants and/or auditory brainstem implants. For example, cochlear implants can provide a person having sensorineural hearing loss with the ability to perceive sound by stimulating the person's auditory nerve via an array of electrodes implanted in the person's cochlea. A component of the cochlear implant detects sound waves, which are converted into a series of electrical stimulation signals that are delivered to the implant recipient's cochlea via the array of electrodes. Auditory brainstem implants can use technology similar to cochlear implants, but instead of applying electrical stimulation to a person's cochlea, auditory brainstem implants apply electrical stimulation directly to a person's brain stem, bypassing the cochlea altogether. Electrically stimulating auditory nerves in a cochlea with a cochlear implant or electrically stimulating a brainstem may enable persons with sensorineural hearing loss to perceive sound. Further, some persons may benefit from hearing prosthesis that combine one or more characteristics of the acoustic hearing aids, vibration-based hearing devices, cochlear implants, and auditory brainstem implants to enable the person to perceive sound.

Each type of hearing prosthesis has an associated sound processor. In one basic hearing prosthesis, the sound processor just provides an amplification to any sounds received by the prosthesis. However, in other hearing prostheses, the processor is more advanced. For example, some processors are programmable and include advanced signal processing functions (e.g., noise reduction functions) and speech algorithms adapted to cochlea electrode stimulation.

In some hearing prosthesis systems, prostheses are present on both the left and right sides of the recipient. The left prosthesis provides audio to the left ear and the right prosthesis provides audio to the right ear. The two prostheses may operate independently of each other. However, it may be desirable for the two prostheses to communicate with one another and even transfer the captured audio from the left ear to the right ear prosthesis and vice versa.

### SUMMARY

As discussed above, a traditional hearing prosthesis system may have two hearing prosthesis devices, one for each ear. Generally, upon receipt of the input signal, each hearing prosthesis uses a microphone to convert an acoustic wave into an electrical signal. Each prosthesis will then provide a stimulation to the recipient of the hearing prosthesis based on the electrical signal. In the case of a cochlear implant (or other prosthesis having an implanted component), the prosthesis may have both an external portion and an internal portion.

The external portion may provide a wireless communication to the internal portion providing a stimulation signal. Additionally, each prosthesis can be configured to communicate with the other prosthesis. A second wireless signal may provide a communication link between the two prostheses. The communication link between the two prostheses allows the prostheses to exchange information. By exchanging information, a better quality stimulation signal may be provided to the recipient of the prosthesis system. For example, a portion of the audio signal received by the left implant may be used to create a stimulus for the right implant.

In order to avoid possible wireless interference when each prosthesis is wirelessly communicating with both a corresponding internal component and the other external component, the disclosed systems and methods operate in a specific communication mode. The communication mode includes transmitting a first signal from a first external component to a first internal component during a first period of time. Additionally, the mode includes transmitting a second signal from the first external component to a second external component during the first period of time. Conversely, during a second period of time, the first external component refrains from transmitting signals to either the first internal component or the second external component.

During the second period of time, the second external component functions in a similar manner as previously discussed with respect to the first external component. The second external component transmits a third signal from the second external component to a second internal component during the second period of time. Additionally, the second external component transmits a fourth signal to the first external component during the second period of time. However, during the first period of time, the second external component refrains from transmitting signals to either the second internal component or the first external component. In some embodiments, the first period of time is equal in length to the second period of time. Additionally, the first signal (and third signal) may be a combined power signal and data signal, whereas the second signal (and fourth signal) may be a data signal.

### BRIEF DESCRIPTION OF THE DRAWINGS

FIG 1A is a block diagram illustrating an example of a hearing prosthesis.

FIG. 1B is a schematic diagram illustrating an example of two cochlear implants coupled via a bilateral wireless link.

FIG. 2 is a block diagram illustrating two cochlear implants coupled via a bilateral wireless link.

FIG. 3 illustrates timing diagrams for use with two cochlear implants coupled via a wireless link.

FIG. 4 is a flow diagram illustring an example method for the timing of transmission with two cochlear implants.

### DETAILED DESCRIPTION

For illustration purposes, some systems and methods are described with respect to cochlear implants. However, many systems and methods may be equally applicable to other types of hearing prostheses. Certain aspects of the disclosed systems and methods could be applicable to any type of hearing prosthesis now known or later developed. Further, some of the disclosed methods can be applied to acoustic devices other than hearing prostheses.

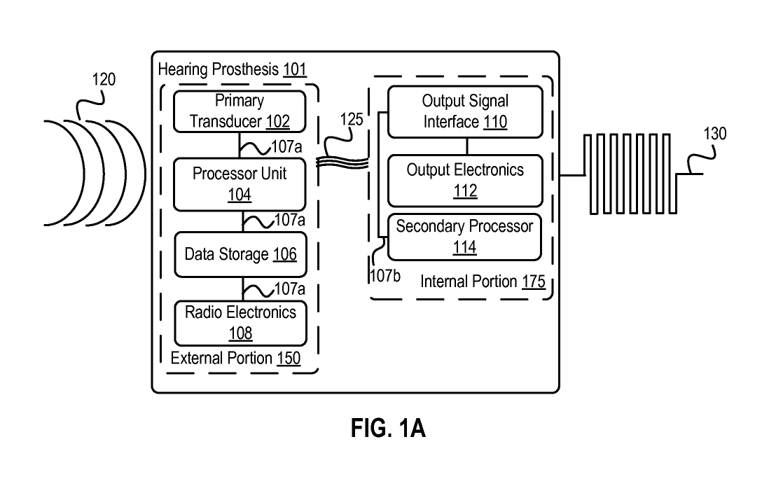

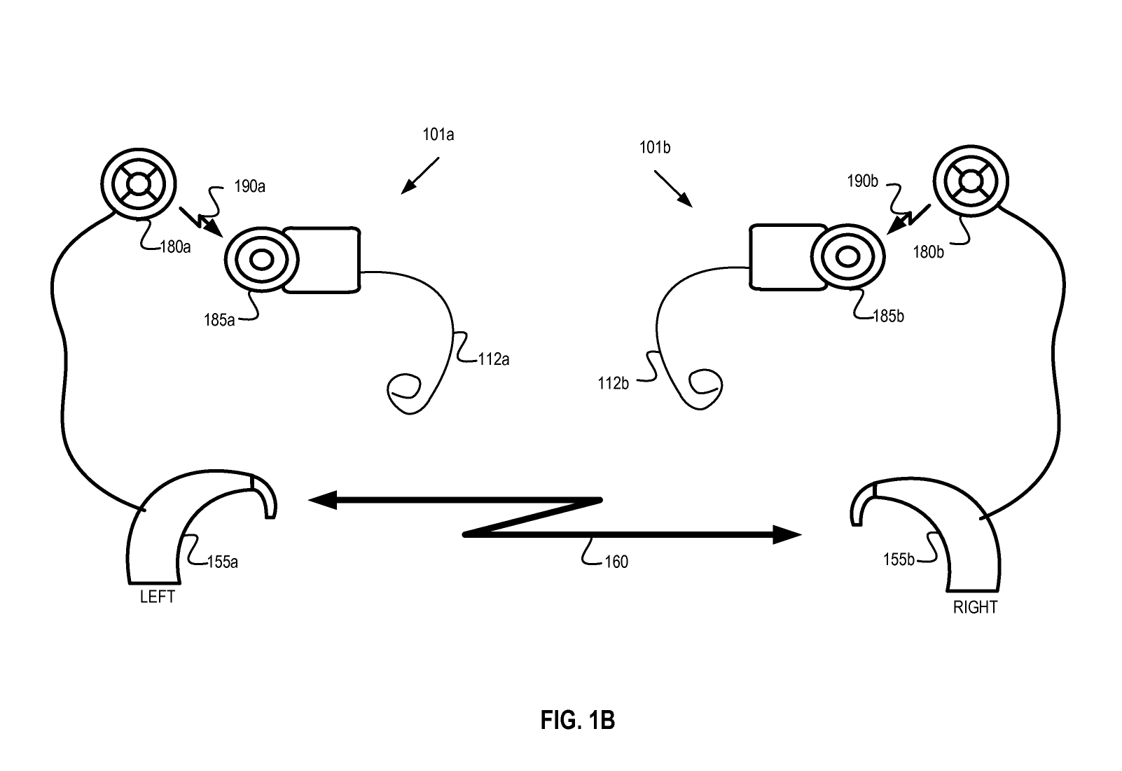

FIG. 1A is a block diagram illustrating one example of a hearing prosthesis 101 configured according to some embodiments of the disclosed systems and methods. The hearing prosthesis 101 may be a cochlear implant, acoustic hearing aid, bone-anchored hearing aid or other vibration-based hearing prosthesis, direct acoustic stimulation device, auditory brain stem implant, or other type of hearing prosthesis configured to receive and process at least one signal from an audio transducer of the prosthesis.

The hearing prosthesis 101 includes an external portion 150 and an internal portion 175. The external portion 150 includes a primary transducer 102, data storage 106, radio electronics 108, and the processor unit having one or more processor units 104, all of which are connected directly or indirectly via circuitry 107a. The internal portion 175 includes an output signal interface 110, output electronics 112, and a secondary processor 114, all of which connect directly or indirectly via circuitry 107b. In other embodiments, the hearing prosthesis 101 has additional or fewer components than the prosthesis shown in FIG. 1. For example, in some embodiments, the external portion 150 includes a secondary transducer. Additionally, the components may be arranged differently than shown in FIG. 1. For example, depending on the type and design of the hearing prosthesis, the illustrated components may be enclosed within a single operational unit or distributed across multiple operational units (e.g., multiple external units and an internal unit). The hearing prosthesis 101 includes radio electronics and one or more processor units 104 configured to process the audio (e.g. by a DSP processor) and to control the interfaces (e.g. by a controller, such as a micro-controller).

In embodiments where the hearing prosthesis 101 is a cochlear implant, the hearing prosthesis comprises an external portion 150 worn outside the body and an internal portion 175 located or implanted within the body. The external portion 150 is coupled to the internal portion 175 via an inductive coupling pathway 125. The primary transducer 102 receives acoustic signals 120, and the processor unit 104 analyzes and encodes the acoustic signals 120 into a group of electrical stimulation signals 130 for application to an implant recipient's cochlea via an output signal interface 110 communicatively connected to output electronics 108.

The external portion 150 is able to power the internal portion 175 via a wireless signal. In some embodiments, the internal portion 175 also has a power source, such as a battery or capacitor, to provide energy to the electronic components housed within the internal portion 175. In this case the external portion 150 is able to inductively charge the power source within the internal portion 175 via a wireless signal. In an example embodiment, a power source that is part of the external portion 150 is the primary power source for the hearing prosthesis. In this example, the power source within the internal portion 175 is only used as a backup source of power. The battery in the internal portion 175 is used as a backup power source when either the external portion 150 runs out of power or when the external portion 150 is decoupled from the internal portion 175.

In some embodiments, the audio source emanates from another separate external portion (not shown). For example, the audio source may be located in a standard computer, a laptop computer, a tablet computing device, a mobile device such as a cellular phone, or a custom computing device. The primary transducer 102 may wirelessly transfer these analog audio signals to the processor unit 104. Further, the external portion 150 may also include a secondary transducer. The secondary transducer may be the same type of transducer as the primary transducer 102. However, in some embodiments, the secondary transducer is a different type of transducer than the primary transducer 102. Additionally, the external portion 150 may contain radio electronics 108. The radio electronics 108 allow the hearing prosthesis 101 to communicate with other hearing prostheses (not shown) near hearing prosthesis 101.

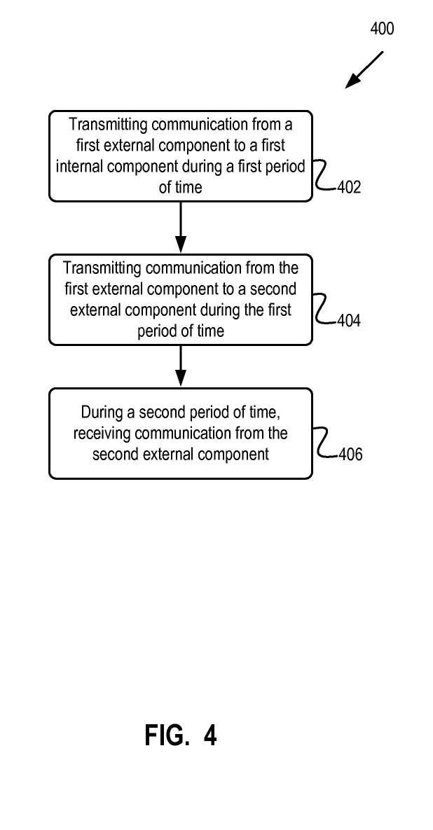

The external portion may also include a data storage 106. The data storage 106 generally includes any suitable volatile and/or non-volatile storage components. Further, the data storage 106 may include computer-readable program instructions and perhaps additional data types. In some embodiments, the data storage 106 stores an amplitude response, a phase response, wireless communication parameters, and recipient-specific parameters associated with the hearing prosthesis. Additionally, the data storage 106 stores a set of signal processing modes and associated parameters for each respective signal processing mode. In other embodiments, the data storage 106 also includes instructions used to perform at least part of the disclosed methods and algorithms, such as method 400 described with respect to FIG. 4. Further, the data storage 106 may be configured with instructions that cause the processor unit 104 to execute functions relating to any of the modules disclosed herein.

For a cochlear implant, the output electronics 112 are connected to an array of electrodes. Individual sets of electrodes in the array of electrodes are grouped into stimulation channels. Each stimulation channel has at least one working electrode (current source) and at least one reference electrode (current sink). During the operation of the prosthesis, the cochlear implant applies electrical stimulation signals to a recipient's cochlea via the stimulation channels. It is these stimulation signals that cause the recipient to experience sound sensations corresponding to the sound waves received by the primary transducer 102 and encoded by the DSP of processor unit 104.

FIG. 1B is a schematic diagram illustrating an example of two cochlear implant devices 101a and 101b coupled via a wireless magnetic induction (MI) link 160. The external portion of the left cochlear implant 101a has a behind-the-ear component 155a and an external coil unit 180a. The internal portion for the left cochlear implant 101a has an internal coil 185a and output electronics 112a. The external coil 180a is typically held in place and aligned with the implanted internal coil 185a via magnets located near each coil. In one embodiment, the external coil 180a is configured to transmit electrical signals to the internal coil 185a via a radio frequency (RF) link 190a. In some embodiments, the external coil 180a is also configured to transmit electrical signals to the internal coil 185a via a magnetic (or inductive) coupling.

Similar to the left cochlear implant 101a, the external portion for the right cochlear implant 101b has a behind-the-ear component 155b and a coil unit 180b. The internal portion for the right cochlear implant 101b has an internal coil 185b and output electronics 112b. Coil 180b of the external portion wirelessly couples to the internal coil 185b via a wireless link 190b. The external coil 180b is typically held in place and aligned with the implanted internal coil 185b via magnets located near each coil. In one embodiment, the external coil 180b is configured to transmit electrical signals to the internal coil 185b via an RF link. In some embodiments, the external coil 180b is also configured to transmit electrical signals to the internal coil 185b via a magnetic (or inductive) coupling.

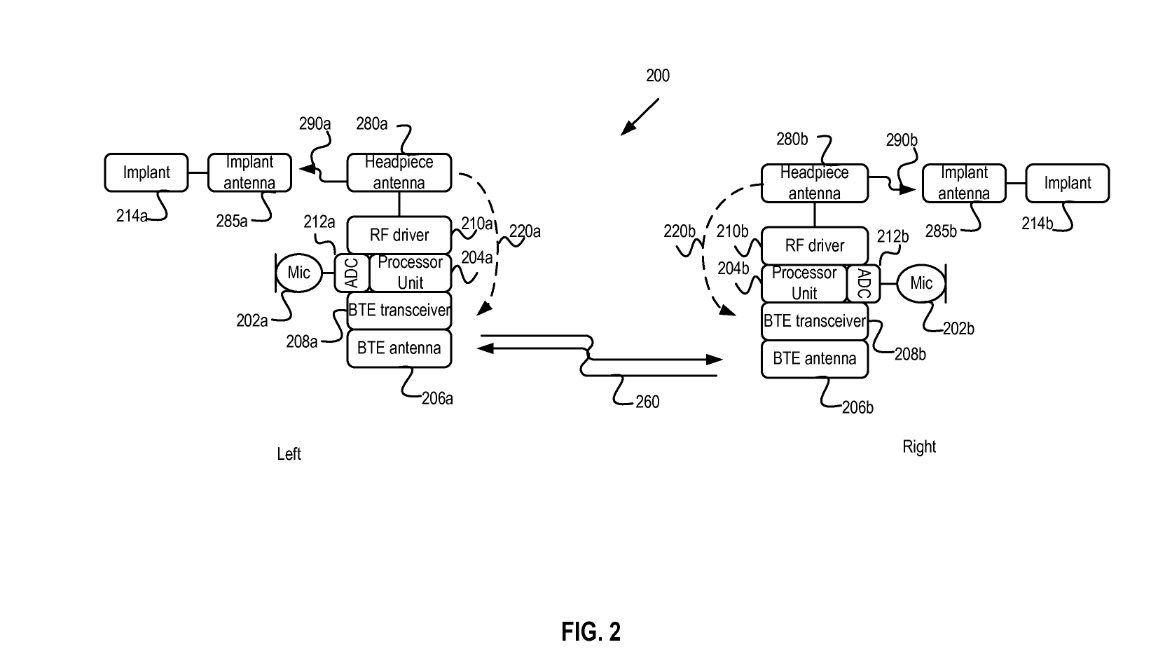

FIG. 2 is a block diagram illustrating a system 200 featuring two cochlear implant devices coupled via a bilateral wireless link 260. System 200 of FIG. 2 is similar to the two cochlear implant devices 101a and 101b of FIG. 1B. Similar to FIG. 1B, FIG. 2 shows a left cochlear implant device and right cochlear implant device coupled via a communication link 260, in this example established by the behind the ear (BTE) transceivers (208a and 208b) through the BTE antennas (206a and 206b).

Both the left and the right cochlear implant devices have an internal portion and an external portion. The devices have internal portions with respective implant antennas 285a and 285b and implant devices 214a and 214b. Additionally, the devices have respective external portions with microphones 202a and 202b, analog-to-digital converters 212a and 212b, processor units 204a and 204b, BTE transceivers 208a and 208b, BTE antennas 206a and 206b, radio frequency (RF) drivers 210a and 210b, and headpiece antennas 280a and 280b.

The headpiece antenna 280a and 280b of each external portion is transcutaneously coupled (i.e. through the skin) with the respective implant antenna 285a and 285b to enable power and data signal links 290a and 290b. An efficient inductive power transfer occurs when the headpiece antenna 280a or 280b and the implant antennas 285a or 285b are planar circular coils placed closely, concentrically, and plane-parallel to the implant antenna. The BTE antenna 206a in the left cochlear implant device may communicate with the BTE antenna 206b in the right cochlear implant device via the bilateral communication link 260 over a larger range, such as 30 cm.

The function of the various blocks is now described with respect to the left cochlear implant device. The blocks of the right cochlear implant device will generally function in a similar manner as described for the left implant device.

Typically, microphone 202a receives a sound wave and converts the sound wave into an electrical signal. The electrical signal is a representation of the audio contained in the received sound wave. In some embodiments, a different form of transducer may replace microphone 202a. However, the functionally will generally be similar.

The output of the microphone 202a is an analog electrical signal that is passed to the analog-to-digital converter 212a. The analog-to-digital converter 212a converts the analog signal from the microphone 202a into a digital representation of the signal. The digital signal from the analog-to-digital converter 212a is passed to the processor unit 204a.

The processor unit 204a contains one or more processors as described with respect to FIG. 1A. In this example the function of the processor unit 204a is twofold: processing the incoming audio signal and controlling the interfaces.

First, the processor unit 204a creates an adapted stimulation signal to provide to the recipient. This specific signal processing is performed by one or more DSP functions. The DSP adapts the digital signal from the analog-to-digital converter 212a before it is applied to the prosthesis recipient. DSP functions transform the digital signal into an output signal by modifying at least one audio attribute of the digital signal to create the output signal. In turn, the audio attributes are modified based on signal processing parameters associated with the DSP of the processor unit 204a. For example, the modified audio attributes may include acoustic gain tables, frequency response curves, and other audio parameters. Further, the DSP functions may be based on a hearing impairment associated with the prosthesis recipient. Once the output signal is created, it is passed to the RF driver 210a.

Second, the processor unit 204a creates a communication signal for timely transmissions and receptions to the external portion of the hearing prosthesis located on the opposite side of the recipient's head (i.e. the right device communicates a signal to the left device). The processor unit 204a makes a determination as to what information should be communicated to the external portion of the opposite hearing prosthesis. This information includes, for example, auditory information, data, and/or status information, such as a timing synchronization. Regardless of what type of information is included in the communication signal, the processor unit 204a creates and outputs the communication signal to the BTE transceiver 208a. In some embodiments, the BTE transceiver 208a (or 208b) is a magnetic induction (MI) transceiver.

The RF driver 210a receives the output signal created by the processor unit 204a and creates a signal to communicate to the headpiece antenna 280a. The output signal received by the RF driver 210 may be a digital modulated signal such as on-off keying (OOK) or frequency-shift keying (FSK). In some embodiments, the RF driver 210a up-converts the frequency of the analog signal before passing it to the headpiece antenna 280a. In some other embodiments, the RF driver 210a increases the amplitude of the signal before passing it to the headpiece antenna 280a. In either instance, the purpose of the RF driver 210a is to condition (boost, convert) the digital output signal from the processor unit 204a into a signal the RF driver 210a may transmit. The RF driver 210a may perform its function differently than disclosed above, depending on the specific embodiment.

Headpiece antenna 280a receives the signal from RF driver 210a and converts it to an electromagnetic signal. In this example, the headpiece antenna 280a may be a wire coil that induces a strong magnetic field when an electric current flows through it. The strong magnetic component of the electromagnetic signal forms the power and data communication link 290a.

The implant antenna 285a and headpiece antenna 280a are preferably coils, so that the implant antenna 285a receives the time-varying magnetic signal from the power and data communication link 290a. The magnetic signal forms the power and data communication link 290a between the headpiece antenna 280a and the implant antenna 285a located underneath the surface of the skin of the prosthesis recipient. The implant antenna 285a converts the received magnetic signal into an electrical signal that is applied to implant 214a.

In some embodiments, the power and data communication link 290a is a two-way link (i.e. headpiece antenna 280a both transmits and receives from implant antenna 285a). The two-way link allows information to be sent from the internal portion to the external portion. In some embodiments, the power and data communication link 290a provides both power and data to the implant antenna 285a. The power portion of power and data communication link 290a may be a radio signal that the implant 214a is able to rectify into a voltage. The rectified voltage is used to charge either a battery or capacitor that powers the implant 214a. Additionally, power and data communication link 290a transmits data to the implant antenna 285a. This transmitted data typically is electrode stimulation information or processed audio that implant 214a uses to create a stimulus for the prosthesis recipient. The implant 214a receives the signal from the implant antenna 285a. Upon receiving the signal, the implant 214a creates an output to be applied to the recipient of the hearing prosthesis. The output of the hearing prosthesis is a current supplied by an electrode (such as the output electronics 112a of FIG. 1B). In some embodiments, the implant may be a hearing prosthesis other than a cochlear implant. In those cases, the output will be a different type of stimulation. For example, some hearing prostheses provide a mechanical stimulation of either the middle or inner ear.

The processor unit 204a outputs a communication signal to the BTE transceiver 208a. The BTE transceiver 208a creates a signal to be transmitted via the BTE antenna 206a. The BTE transceiver 208a receives the output signal created by the processor unit 204a and creates a signal to communicate to the hearing prosthesis unit on the opposite side of the head. The output signal received by the BTE transceiver 208a may be a digital signal, in which case, the BTE transceiver 208a performs a digital-to-analog conversion. In some embodiments, the BTE transceiver 208a up-converts the frequency before passing it to the BTE antenna 206a. In other embodiments, the BTE transceiver 208a transmits a digital signal to the BTE antenna 206a. In yet other embodiments, the BTE transceiver 208a increases the amplitude of the signal before passing it to the BTE antenna 206a. The purpose of the BTE transceiver 208a is to convert the digital output signal from the processor unit 204a into a signal that the BTE antenna 206a transmits in a timely and repetitive way. The BTE transceiver 208a may perform its function differently than disclosed above, depending on the specific embodiment.

The BTE antenna 206a is preferably a wire coil that receives a signal from the BTE transceiver 208a and induces a magnetic signal when an electric current flows through it or generates a voltage when placed in a time varying magnetic field. The electromagnetic signal created by the BTE antenna 206a may be a magnetic field or a radio wave. The BTE antenna 206a may take many different forms, depending on the specific embodiment. For example, the BTE antenna 206a may be a loop antenna, such as a magnetic loop or MI (magnetic induction) antenna. In other embodiments, BTE antenna 206a may be a patch antenna.

The BTE antenna 206a is also configured to receive incoming signals over bilateral communication link 260 from the corresponding BTE antenna 206b of the cochlear implant device located on the other side of the head. When the BTE antenna 206a receives a signal from the bilateral communication link 260, the BTE antenna 206a passes the signal to the BTE transceiver 208a. The BTE transceiver 208a receives the signal and creates a signal to communicate to the DSP of processor unit 204a. When operating in a receiving mode, the BTE transceiver 208a demodulates the received signal into a digital signal. In some embodiments, before digitization, the BTE transceiver 208a down-converts the frequency of the received signal to a base-band audio signal prior to demodulation and digitization.

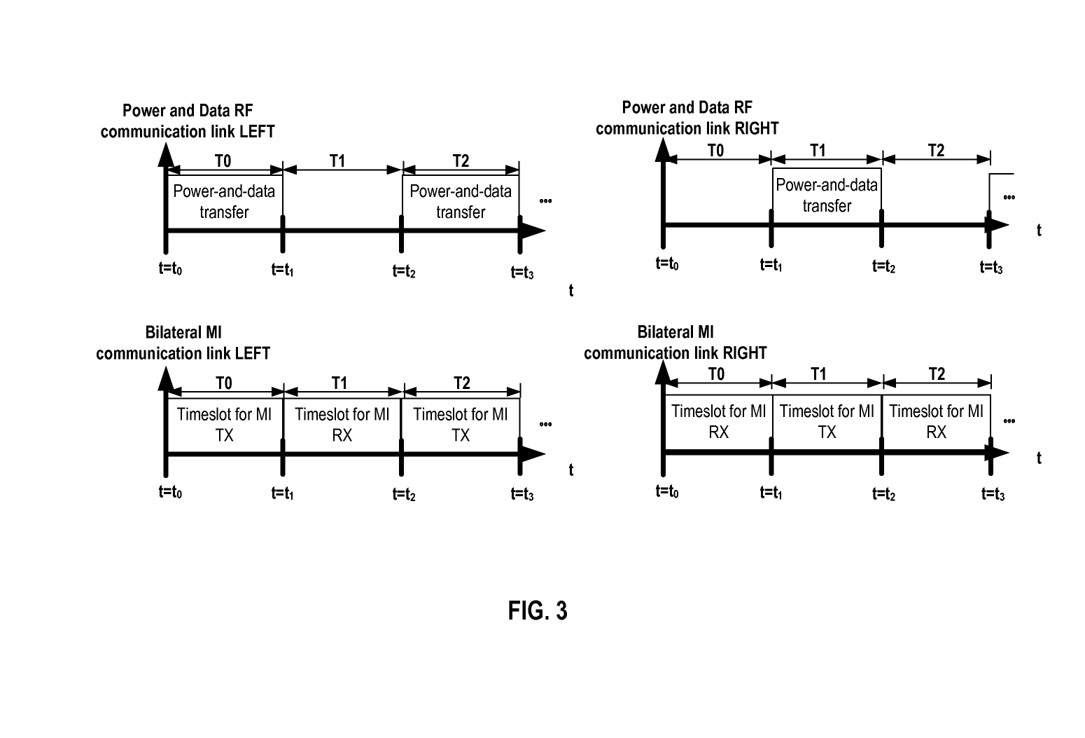

In accordance with some embodiments, the system 200 operates in a specific communication mode, set forth in further detail with respect to FIGS. 3 and 4. Under this specific communication mode, the left-side prosthesis only transmits with the headpiece antenna 280a when the BTE transceiver 208a is not in the receive mode. Further, the right-side prosthesis transmits with the headpiece antenna 280b when the BTE transceiver 208b is not in the receive mode. Operating the system 200 in such a mode helps to mitigate potential wireless interference that might otherwise result due to each prosthesis communicating with both a corresponding internal component and the other external component. More specifically, this potential wireless interference could result if the BTE antenna 206a were to also receive an interference signal 220a from the transmission of the headpiece antenna 280a, based on the power and data communication link 290a. For example, when the headpiece antenna 280a is a coil that transfers power and data to the implant antenna 285a wirelessly over power and data communication link 290a, a portion of the signal from the power and data communication link 290a could be coupled to the BTE antenna 206a via the interference signal 220a. Due to the relatively close proximity of the headpiece antenna 280a to the BTE antenna 206a, as compared to the distance between the BTE antenna 206a and the BTE antenna 206b, the interference signal 220a may suffer less attenuation than the bilateral communication link 260 does. Thus, the BTE transceiver 208a may not be able to distinguish the bilateral communication link 260 from the interference signal 220a if the specific communication mode set forth herein were not used.

FIG. 3 is an example timing diagram 300 illustrating a timing scheme for a system having two cochlear implants coupled via a wireless link, such as the system 200 of FIG. 2. Operating according to the timing scheme shown in FIG. 3 mitigates wireless interference in the cochlear implant system. As shown in FIG. 3, the bilateral communication link LEFT and bilateral MI communication link RIGHT represent communication link 260 of FIG. 2. Additionally, Power and Data RF communication link LEFT represent power and data communication links 290a of FIG. 2. Further, Power and Data RF communication link RIGHT represent power and data communication links 290b of FIG. 2. With each prosthesis operating in either a transmit or receive mode, the system mitigates the impact of the interference signals 220a and 220b on the communication link 260.

As shown in FIG. 3, there are three representative time slots. Time slot T0 runs from t₀ to t₁, time slot T1 runs from t₁ to t₂, and time slot T2 runs from t₂ to t₃. FIG. 3 shows the specific signals that are being transmitted (TX) or received (RX) by the left and right cochlear implants during any given time period. Further, FIG. 3 also shows when specific signals are not being transmitted. Additionally, as illustrated in FIG. 3, during any given time period, one of the two prostheses is in a transmit mode and the other prosthesis is in a receive mode.

At time slot T0, two signals are being transmitted in the cochlear implant system. During time slot T0, only the left cochlear implant is transmitting any signals. In fact, the left cochlear implant transmits two signals simultaneously during time slot T0. A Power and Data RF communication link LEFT is being transmitted from a headpiece antenna to an implant antenna, such as the headpiece antenna 280a and implant antenna 285a of FIG. 2. Additionally, a Bilateral communication link LEFT is transmitted from one cochlear implant and received by the other cochlear implant as Bilateral communication link RIGHT. In one example, BTE antenna 206a of FIG. 2 transmits Bilateral communication link LEFT along communication link 260 of FIG. 2. Communication link 260 of FIG. 2 is received by BTE antenna 206b as Bilateral communication link RIGHT. Further, during time slot T0, there is no signal sent as Power and Data RF communication link RIGHT.

At the transition from time slot T0 to time slot T1, the signaling switches. The prosthesis that was transmitting during time slot T0 goes into a receive mode and the prosthesis that was receiving during time slot T0 goes into a transmit mode.

At time slot T1, two signals are being transmitted in the cochlear implant system. During time slot T1, only the right cochlear implant is transmitting any signals. The right cochlear implant transmits two signals simultaneously during time slot T1. A Power and Data RF communication link RIGHT is being transmitted from a headpiece antenna to an implant antenna, such as the headpiece antenna 280b and implant antenna 285b of FIG. 2. Additionally, a Bilateral communication link RIGHT is transmitted from one cochlear implant and received by the other cochlear implant as Bilateral communication link LEFT. In one example, the BTE antenna 206b of FIG. 2 transmits Bilateral communication link RIGHT along communication link 260 of FIG. 2. Communication link 260 of FIG. 2 is received by BTE antenna 206a as Bilateral communication link LEFT. Further, during time slot T1, there is no signal sent as Power and Data RF communication link LEFT.

Similar to the transition from time slot T0 to time slot T, at the transition from time slot T1 to time slot T2, the signaling again switches. The prosthesis that was transmitting during time slot T1 goes into a receive mode and the prosthesis that was receiving during time slot T1 goes into a transmit mode. The signaling during time slot T2 may be the same as time slot T0.

At time slot T2, two signals are being transmitted in the cochlear implant system shown in FIG. 3. During time slot T2, only the left cochlear implant is transmitting signals. The left cochlear implant transmits two signals simultaneously during time slot T2. A Power and Data RF communication link LEFT is being transmitted from a headpiece antenna to an implant antenna, such as the headpiece antenna 280a and implant antenna 285a of FIG. 2. Additionally, a Bilateral communication link LEFT is transmitted from one cochlear implant and received by the other cochlear implant as Bilateral communication link RIGHT. In one example, BTE antenna 206a of FIG. 2 transmits Bilateral communication link LEFT along communication link 260 of FIG. 2. Communication link 260 of FIG. 2 is received by BTE antenna 206b as Bilateral communication link RIGHT. Further, during time slot T2, there is no signal sent as Power and Data RF communication link RIGHT.

The hearing prosthesis system may continue to switch the prostheses between transmit and receive modes based on the time slots. In some embodiments, each time slot is approximately the same length of time as every other time slot. However, in some embodiments, it may be advantageous for the prosthesis system to vary the length of the various time slots.

Further, in some embodiments, the processor unit 104 (of FIG. 1) includes an initiation routine. During the initiation routine, the two prostheses communicate (via communication link 260 of FIG. 2) to establish the timing for the time slots. The initiation may also be known as a timing synchronization. The timing synchronization may include a clock signal configured to synchronize the timing between the prostheses. Further, the synchronization may also specify the duration of the time slots.

FIG. 4 is an example method 400 for the timing of transmission with two cochlear implants. As part of method 400, at block 402 a processor in a first external portion of a cochlear implant causes radio electronics in the prosthesis to transmit communication from the first external component to a first internal component during a first period of time. The communication to the internal component is a communication that the internal component uses to (i) rectify into a voltage to supply a power source and (ii) provide data that the first internal component converts into a stimulation to provide to a recipient of the prosthesis.

At block 404, the processor in a first external portion of a cochlear implant causes radio electronics in the prosthesis to transmit communication from the first external component to a second external component during a first period of time. The communication to the second external component may be data related to an audio signal received by the first external portion. The second external portion may use this data in its processing system to create a stimulus for a recipient of the prosthesis.

At block 406, the processor in a first external portion of a cochlear implant causes radio electronics in the prosthesis to receive communication from the second external component during a second period of time. The communication to the second external component may be data related to an audio signal received by the second external portion. The first external portion may use this data from the second external portion in its processing system to create a stimulus for a recipient of the prosthesis.

Further, during the second period of time, a processor in a second external portion of a cochlear implant may cause radio electronics in the prosthesis to transmit communication from the second external component to a second internal component during the second period of time. The communication to the internal component may be a communication that the internal component uses to (i) rectify into a voltage to supply a power source and (ii) provide data that the second internal component converts into a stimulation to provide to a recipient of the prosthesis.

Further, during the second period of time, the first external component may be configured to not transmit any signals. Thus, during the second period of time, the first external component is in a receive mode and the second external component is in a transmit mode. During the first period of time, the second external component is in a receive mode (i.e. not transmitting any signals) and the first external component is in a transmit mode. The method may be performed repeatedly over a plurality of time periods. For each time period, one external component is in a transmit mode while the other external component is in a receive mode. As the time periods advance, the external components simultaneously switch modes.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A method comprising:
transmitting a first signal from a first external component of a hearing prosthesis to a first internal component of the hearing prosthesis during a first period of time;
transmitting a second signal from the first external component to a second external component of the hearing prosthesis during the first period of time, wherein the first and second signals are transmitted simultaneously during the first period of time; and
during a second period of time, the first external component refraining from transmitting signals to either the first internal component or the second external component, wherein the second period of time does not overlap with the first period of time.

2. The method of claim 1, further comprising:
transmitting a third signal from the second external component to a second internal component of the hearing prosthesis during the second period of time;
transmitting a fourth signal from the second external component to the first external component during the second period of time; and
during the first period of time, the second external component refraining from transmitting signals to either the second internal component or the first external component.

3. The method of claim 1, wherein the first period of time is equal to the second period of time.

4. The method of claim 1, wherein the first signal provides power to the first internal component.

5. The method of claim 4, wherein the first signal provides data to the first internal component.

6. The method of claim 2, wherein the third signal provides power to the second internal component.

7. The method of claim 6, wherein the third signal provides data to the second internal component.

8. The method of claim 2, wherein the second signal comprises a data signal, and wherein the second external component uses the data signal to generate the third signal.

9. The method of claim 2, wherein the fourth signal comprises a data signal, and wherein the first external component uses the data signal to generate a fifth signal that the first external component transmits to the first internal component.

10. A system comprising:
a first implantable component of a hearing prosthesis;
a second implantable component of the hearing prosthesis;
a first external component of the hearing prosthesis configured to:
transmit a first signal to the first implantable component via a first wireless link during a first time period,
transmit a second signal to a second external component of the hearing prosthesis via a second wireless link during the first time period, and
during a second time period, refrain from transmitting both (i) the first signal and (ii) the second signal, wherein the second time period does not overlap with the first time period; and
the second external component configured to:
transmit a third signal to the second implantable component via a third wireless link during the second time period,
transmit a fourth signal to the first external component via a fourth wireless link during the second time period, and
during the first time period, refrain from transmitting both (i) the third signal and (ii) the fourth signal.

11. The system of claim 10, wherein the first wireless link operates at a first frequency and the second wireless link operates at a second frequency, wherein the first frequency and the second frequency are different frequencies.

12. The system of claim 11, wherein the third wireless link operates at a third frequency and the fourth wireless link operates at a fourth frequency, wherein the third frequency and the fourth frequency are different frequencies.

13. The system of claim 12, wherein the first and third wireless links operate at the same frequency.

14. The system of claim 12, wherein the second and fourth wireless links operate at the same frequency.

15. The system of claim 10, wherein the first time period is equal to the second time period of time.

16. The system of claim 10, wherein the first external component includes a first coil configured to transmit the first signal via the first wireless link, and wherein the second external component includes a second coil configured to transmit the third signal via the third wireless link.

17. The system of claim 16, wherein the first external component includes a third coil configured to transmit the second signal via the second wireless link, and wherein the second external component includes a fourth coil configured to transmit the fourth signal via the fourth wireless link.

18. The system of claim 10, wherein at least one of the first signal and the second signal is transmitted for less than or equal to a duration of the first time period.

19. The system of claim 10, wherein at least one of the third signal and the fourth signal is transmitted for less than or equal to a duration of the second time period.

20. The system of claim 10, wherein the first implantable component receives power over the first wireless link.

21. The system of claim 10, wherein the second implantable component receives power over the third wireless link.

22. A method comprising:
transmitting a synchronization signal from a first external component to a second external component, wherein the synchronization signal indicates the start of a first time period, and wherein the first external component and the second external component are configured to be worn outside a body of a recipient;
during the first time period:
transmitting a data signal from the first external component to the second external component, wherein the data signal is related to an audio signal received by the first external component; and
transmitting a combined signal from the first external component to a first internal component, wherein the first internal component is configured to be implanted in the body of the recipient; and
during a second time period that does not overlap with the first time period, refrain_{g} from transmitting both (i) the data signal and (ii) the combined signal.

23. The method of claim 22, wherein the first synchronization signal indicates the duration of the first time period.

24. The method of claim 22, wherein the combined signal comprises a second data signal and a power signal.

25. The method of claim 22, wherein the data signal is transmitted at a first frequency and the combined signal is transmitted at a second frequency, wherein the first frequency and the second frequency are different frequencies.

26. The method of claim 22, further comprising
during the second time period:
transmitting a second data signal from the second external component to the first external component; and
transmitting a second combined signal from the second external component to a second internal component, wherein the second internal component is configured to be implanted in the body of the recipient; and
during the first time period, refraining from transmitting both (i) the second data signal and (ii) the second combined signal.

27. The method of claim 26, wherein the first combined signal comprises a third data signal and a first power signal, and wherein the second combined signal comprises a fourth data signal and a second power signal.

28. The method of claim 26, wherein the first data signal is transmitted at a first frequency and the first combined signal is transmitted at a second frequency, wherein the first frequency and the second frequency are different frequencies, and wherein the second data signal is transmitted at a third frequency and the second combined signal is transmitted at a fourth frequency, wherein the third frequency and the fourth frequency are different frequencies.

29. A system comprising:
a first external component of a hearing prosthesis configured to:
transmit a synchronization signal indicating the start of a first time period from the first external component to a second external component of the hearing prosthesis,
transmit a first combined signal to a first implantable component of the hearing prosthesis during a first time period, wherein the first combined signal comprises both a first data signal and a first power signal,
transmit a second data signal to the second external component during the first time period, and
during a second time period, refrain from transmitting both (i) the first combined signal and (ii) the second data signal, wherein the second time period does not overlap with the first time period; and
the second external component configured to:
receive the synchronization signal from the first external component,
transmit a second combined signal to a second implantable component of the hearing prosthesis during the second time period, wherein the second combined signal comprises both a third data signal and a second power signal,
transmit a fourth data signal to the first external component during the second time period, and
during the first time period, refrain from transmitting both (i) the second combined signal and (ii) the fourth data signal.

30. The system of claim 29, wherein the first combined signal is transmitted at a first frequency and the second data signal is transmitted at a second frequency, wherein the first frequency and the second frequency are different frequencies.

31. The system of claim 30, wherein the second combined signal is transmitted at a third frequency and the fourth data signal is transmitted at a fourth frequency, wherein the third frequency and the fourth frequency are different frequencies.

32. The system of claim 31, wherein the first and third frequencies are the same frequency.

33. The system of claim 32, wherein the second and fourth frequencies are the same frequency.

34. The system of claim 29, wherein the first time period is equal to the second time period.

35. The system of claim 29, wherein the synchronization signal specifies a duration of the first time period.

36. The system of claim 29, wherein the synchronization signal comprises a clock signal.

37. The system of claim 29, wherein at least one of the first combined signal and the second data signal is transmitted for less than or equal to a duration of the first time period.

38. The system of claim 29, wherein at least one of the second combined signal and the fourth data signal is transmitted for less than or equal to a duration of the second time period.
